(19) United States
(12) Patent Application Publication
(10) Pub. No.: US 2008/0072337 A1
(43) Pub. Date: Mar. 20, 2008

(54) Braf Expression in Zebrafish and Uses Thereof

(75) Inventors: Leonard I. Zon, Wellesley, MA (US); E. Elizabeth Patton, Oxford (GB); E. David Fisher, Newton, MA (US); Hans Widlund, Boston, MA (US)

Correspondence Address: DAVID S. RESNICK, 100 SUMMER STREET, NIXON PEABODY LLP, BOSTON, MA, 02110-2131, US

(73) Assignee: Children's Medical Center Corporation, Boston, MA (US)

(21) Appl. No.: 11/596,146

(22) PCT Filed: May 9, 2005
(86) PCT No.: PCT/US05/15987

§ 371 Date: Nov. 6, 2007

Related U.S. Application Data
(60) Provisional application No. 60/569,732, filed on May 10, 2004.

(51) Int. Cl. A01K 67/00; G01N 33/00

(52) U.S. Cl. 800/3; 800/10

(57) ABSTRACT

The present invention discloses a transgenic zebrafish that express activated BRAF specifically in melanocytes and its uses in screening for agents that can be used to treat melanomas or screening for agents that aggravate or induce melanomas.

### CROSS-REFERENCE TO RELATED APPLICATIONS

The present application claims the benefit under 35 U.S.C. 119 (e) of the U.S. provisional Patent Application No. 60/569,732, filed May 10, 2004, the contents of which is herein incorporated by reference in its entirety.

### BACKGROUND

The skin is the largest organ in the body. It covers and protects the organs inside the body. It also protects the body against germs and prevents the loss of too much water and other fluids. The skin sends messages to the brain about heat, cold, touch, and pain. The skin has 3 layers. From the outside in, they are: the epidermis, the dermis, and the subcutis.

The top layer of the skin, the epidermis, is very thin and serves to protect the deeper layers of skin and the organs. The epidermis itself has three layers: an upper, a middle, and a bottom layer composed of basal cells. These basal cells divide to form keratinocytes, (also called squamous cells) which make a substance (keratin) that helps protect the body. Another type of cell, melanocytes, is also present in the epidermis. These cells produce the pigment called melanin. Melanin gives the tan or brown color to skin and helps protect the deeper layers of the skin from the harmful effects of the sun. A layer called the basement membrane separates the epidermis from the deeper layers of skin.

Melanoma is a very serious form of skin cancer. It begins in melanocytes- cells that make the skin pigment called melanin. The number of new melanomas diagnosed in the United States is increasing. Since 1973, the incidence rate for melanoma (the number of new melanomas diagnosed per 100,000 people each year) has more than doubled from 5.7 to 14.3. Cancer of the skin is the most common of all cancers. Melanoma accounts for about 4% of skin cancer cases, but it causes about 79% of skin cancer deaths. The number of new cases of melanoma in the United States is on the rise. The American Cancer Society estimates that in 2004 there will be 55,100 new cases of melanoma in this country. About 7,910 people will die of this disease. (American Cancer Society Web site at http://www.cancer.org.)

The currently available treatment options for melanoma include surgery, chemotherapy, radiation therapy, as well as in some cases immunotherapy which includes the use of, for example interferon-alpha and interleukin-2 (see, e.g., NCCN Melanoma Treatment Guidelines For Patients at http://www.cancer.org). However, the initial surgical resection is the preferred treatment method of choice, but its success largely depends on early detection. Because melanoma shows early metastasis, it is critical to remove the tumor early because the later stage, distally, melanomasa have poor prognosis and there are currently no effective treatment options available for these patients.

Recently, activating mutations in BRAF, a serine/threonine kinase that transduces RAS regulatory signals, have been found in the majority of nevi and melanomas. The mutant BRAF kinases are constantly active and do not require upstream regulation. Melanoma can arise in dysplastic nevi, and activating mutations in BRAF have been recently implicated in the development of nevi (Pollock et al., 2003; Davies et al., 2002).

Functional ascertation of the activated oncogene in an animal model which is able to closely replicate human disease would be advantageous for drug discovery and mechanistic studies of gene cooperation. In the case of melanomas, such a model is lacking today and has proven hard to develop in mice.

Mice offer some advantages as a model organism for the study of cancer genes in general. Many homologues of the cloned human tumor suppressor genes have been mutated in the mouse (McClatchey, A., et al., Curr Opin Genet Develop, 8:304-310, 1998). By obtaining strains carrying germline disruptions of these genes, both the heterozygous and homozygous phenotypes can be studied. Mice having heterozygous loss-of-function mutations represent models of humans with familial cancer syndromes and can serve as a model system for study of the progression of cancer. Additionally, the homozygous mutants can reveal developmental roles of these tumor suppressor genes. The generation of mouse strains with combinations of tumor suppressor gene mutations provides information about the genetic interactions in tumorigenesis. Transgenic mice expressing oncogenes provide information about the effects these genes have on proliferation and differentiation (Eva A., Semin Cell Bio, 3:137-45, 1992). For example, MT/ret transgenic mice expressing the ret oncogene fused to the metallothionein promoter has been proposed as a melanoma study model. However, these mice develop vitiligo and no visible tumors (Lengagne R., et al., Cancer Res. 2004 Feb 15;64(4):1496-501). Moreover, mice are not ideal animals for developing a large scale screen for agents to treat melanoma as the number of mice needed for such screen is difficult and costly to maintain (Hrabe de Angelis M. et al., Mutat Res, 400:25-32, 1998).

In addition, an albino rabbit injected with human uveal melanoma cells appears to develop uveal melanoma in the eye (Morilla-Grasa a, Cassie AL, Lopez R, et al. Animal model primary and metastatic human uveal melanoma: Co-expression of vimentin and cytokeratin by melanoma cells with different metastatic potential (abstract), Invest Ophthalmol Visual Sci 2001; 42:S217, B479; Lewandowski E, Blanco P L, Caissie A L, Morilla-Grassa A, Colls-Lartigue J J, Bumier M N Jr. Metastatic behaviour of human uveal melanoma cell lines in a rabbit model [abstract]. Invest Ophthalmol Visual Sci 2003; 44:1569, B465). However, rabbit model suffers from the same problem of being expensive and difficult to maintain for the purposes of large scale screening of agents to treat melanomas.

Therefore, it would be useful to develop an animal model which closely resembles histological as well as pathological behavior of human melanoma using bonafide melanoma-associated oncogenes tumor-supressors. Such a model would provide a tool to a robust and relatively inexpensive screen for a large amount of candidate agents for treatment of melanomas. It would also be beneficial to develop a model for nevi, as the benign nevi often serve as the initial event in formation of melanoma. The nevus model could be used to study the melanoma triggering factors as well as treatments to prevent the conversion from nevi to melanoma.

### SUMMARY

Accordingly, the present invention is directed to a transgenic zebrafish that express activated BRAF specifically in melanocytes and its use in screening for agents that can be used to treat melanomas or screening for agents that aggravate or induce melanomas.

The invention is based upon our findings that activated mutant human BRAF, but not wild type BRAF, was able to induce highly visible, ectopic nevi, also known as moles, in a transgenic zebrafish. The mutant human BRAF induced fish- nevi (“f-nevi”) represent a proliferation of melanocytes and are not neoplastic. The invention is further based upon the surprising finding that when activated BRAF is expressed in p53 deficient zebrafish, the fish developed an aggressive, invasive melanoma. The zebrafish melanomas can be serially transplanted. Histological analysis shows high similarity between zebrafish and human melanomas, making this the first solid tumor model in the zebrafish. Therefore, the present invention establishes a melanoma model in zebrafish, provides the first zebrafish example of a genetic interaction promoting cancer, and is the first report to demonstrate a BRAF function and genetic interaction in vivo.

Accordingly, in one embodiment, the invention provides a transgenic zebrafish that expresses, in the fish melanocytes, mutant human BRAF protein.

In one embodiment, the human BRAF protein comprises one or more mutations in its kinase domain.

In one embodiment, the human BRAF protein is encoded in the zebrafish by a construct comprising a human BRAF protein encoding sequence operably linked to a melanocyte specific promoter. In one preferred embodiment, the melanocyte promoter is nacre.

In another embodiment, the invention provides a transgenic zebrafish expressing a combination of a mutant human BRAF protein and a mutant tumor suppressor protein. In one preferred embodiment, the tumor suppressor protein is mutant p53. In the most preferred embodiment, the mutant p53 lacks exon 7.

In one embodiment, the invention provides any one of the above described transgenic zebrafish, wherein expression of the expression product(s) is stable and transmitted through the germline.

In one embodiment, the invention provides a method for identifying a compound that can facilitate mole regression comprising administering a test compound or agent or physical condition to a transgenic zebrafish, which has been genetically modified to express a nucleic acid encoding a mutant human BRAF protein, wherein said mutant human BRAF encoding protein comprises a mutation in the BRAF kinase domain, and wherein the mutant human BRAF protein encoding nucleic acid is under a melanocyte-specific promoter thereby resulting in a zebrafish which develops visible nevi as an adult zebrafish, wherein reduction in the size and/or number of the nevi on the zebrafish skin after exposure to the test agent or test physical condition compared to a transgenic fish that has not been exposed to the test agent indicates that the test agent can facilitate mole regression.

In another embodiment, the invention provides a method for identifying a compound that can facilitate inhibition of melanoma growth comprising administering a test compound to a melanoma model zebrafish, which has been genetically modified to express a nucleic acid encoding a proto-oncogene and a nucleic acid encoding a mutant human BRAF protein wherein said BRAF encoding protein comprises a mutation in the BRAF kinase domain and wherein the mutant human BRAF protein encoding nucleic acid is under a melanocyte-specific promoter, said fish developing visible melanomas in an adult fish, wherein reduction of the size and/or the number of the visible melanoma growth and/or inhibition of the melanoma cell proliferation rate and/or regression of the melanoma cells into nevi after zebrafish exposure to the test compound compared to a similar zebrafish not exposed to the test compound is indicative of identification of a compound can facilitate inhibition of melanoma growth.

In yet another embodiment, the invention provides a method for screening for compounds or agents or physical conditions that can inhibit conversion from nevi into melanoma comprising administering a test agent to a melanoma model zebrafish, which has been genetically modified to express a nucleic acid encoding a proto-oncogene and a nucleic acid encoding a mutant human BRAF protein, wherein said BRAF encoding protein comprises a mutation in the BRAF kinase domain and wherein the mutant human BRAF protein encoding nucleic acid is under a melanocyte-specific promoter said fish developing visible melanomas in an adult fish, wherein inhibition of the conversion from nevi to melanoma is indicative of the test agent having the ability to prevent the nevi from converting to melanoma.

Conversely, the invention also provides methods that can be used to screen for agents that induce, or aggravate melanoma formation. Such methods can be used to create fish that can then be used to screen for agents or physical conditions that counter their effect.

Accordingly, the invention provides a method for screening for a tumor-promoting agent or physical treatment with an ability to promote mole formation comprising administering a test agent to a zebrafish, which has been genetically manipulated to express a nucleic acid encoding a mutant human BRAF protein wherein said BRAF encoding protein comprises a mutation in the kinase domain and wherein the mutant human BRAF protein encoding nucleic acid is under a melanocyte-specific promoter thereby resulting in a zebrafish which develops visible nevi as an adult zebrafish, wherein increase in the size and/or number of the nevi on the zebrafish skin after exposure to the test agent indicates that the test agent has the ability to promote mole formation.

In yet another embodiment, the invention provides a method for screening for a tumor-promoting agent or physical treatment with an ability to promote melanoma growth comprising administering a test agent to a melanoma model zebrafish, which has been genetically modified to express a nucleic acid encoding a mutant proto-oncogene and a nucleic acid encoding a mutant human BRAF protein wherein said BRAF encoding protein comprises a mutation in the kinase domain and wherein the mutant human BRAF protein encoding nucleic acid is under a melanocyte-specific promoter, said fish developing visible melanomas in an adult fish, wherein increase of the size and/or the number of the visible melanoma growth and/or promotion of the melanoma cell proliferation rate and/or progression of the melanoma cells into a more invasive or malignant state after exposure to the test agent indicates that the agent has the ability to promote melanoma growth.

In one embodiment, the invention also provides a method for screening for a tumor-promoting agent or physical treatment with the ability to promote conversion from nevi into melanoma comprising administering a test agent to a melanoma model zebrafish, which has been genetically modified to express a nucleic acid encoding a mutant proto-oncogene and a nucleic acid encoding a mutant human BRAF protein wherein said BRAF encoding protein comprises a mutation in the kinase domain and wherein the mutant human BRAF protein encoding nucleic acid is under a melanocyte-specific promoter said fish developing visible melanomas in an adult fish, wherein promotion of the conversion from nevi to melanoma is indicative of the test agent having the ability to promote the nevi converting to melanoma.

### BRIEF DESCRIPTION OF FIGURES

FIGS. 1A-1C demonstrate that BRAF^{V599E} (also called BRAF^{V600E} in the literature) induces f-nevi in adult zebrafish. Single cell embryos, from FIG. 1A wildtype, or FIG. 1B leopard genetic backgrounds were injected with BRAF^{V599E} and monitored for ectopic melanocytic proliferations, or f-nevi (asterisks). Top fish are control siblings with normal pigmentation patterns, while bottom fish display ectopic f-nevi. FIG. 1C shows that stable expression of BRAF^{V599E} from the mitfa promoter (lower panel) produces ectopic melanocytes on the dorsal side of the fish, widening the second most posterior adult stripe, and almost fusing with the narrow top stripe, compared to wildtype fish (upper panel).

FIGS. 2A-2F show histology of f-nevi. FIG. 2A show adult fish that were sectioned through the melanocytic lesion or f-nevus (dashed line) and stained with hematoxylin and eosin stain with 100× magnification shown in FIG. 2B. Note the left eye, that had been marked with a f-nevus, contains an expansion of melanocytes (arrow), in contrast to the right, normal eye. FIG. 2C shows that F-nevi contain clusters of melanocytes, abundant with black pigment. Sections stained with hematoxylin and eosin are shown at 400× magnification, and FIG. 2D shows 1000× magnification of the same section. FIG. 2E shows comparison of the cellular composition of an f-nevus, and FIG. 2F shows human blue nevus. Like the f-nevus (left), the blue nevus (right) contains numerous elongated, heavily pigmented melanocytes.

FIGS. 3A-3F show melanoma in zebrafish. FIG. 3A shows an AB fish homozygous for p53−/− rapidly develops melanoma over a 10-day period at the site of a BRAF^{V599E} induced f-nevus. F-nevi are seen in the tail, body and dorsal fin at 4 months of age (asterisks; top image). Within two days, the tail of the same fish whitens (middle), and within 4 days has developed small tumors on the tail (not shown). By day 10 a large tumor mass on the fish is clearly visible (arrow, bottom image). FIG. 3B shows hematoxylin and eosin stain of the tumor which shows densely cellular, mitotically active, melanocytic tumor invading the muscle tissue of the tail at 100× and FIG. 3C shows a 400× magnification of the same staining. FIG. 3D shows electron micrographs confirm the presence of melanocytes (arrow) within the tumor, and FIG. 3E shows premelanosomes within the melanocyte (arrow). FIG. 3F shows Western blot analysis which shows the presence of myc-tagged BRAF^{V599E} specifically within the tumor, while normal BRAF and tubulin is detected in normal embryo extract and human tumors.

FIGS. 4A-4F show melanoma characterization. FIG. 4A shows an adult irradiated recipients develop metastatic melanoma visible through the abdomen (asterisks; top & middle fish), and upon gross examination after sagittal sectioning and fixation (bottom fish). FIG. 4B shows the characteristics of the malignant transplanted tumors including invasion of the liver (100×), and FIG. 4C demonstrates aneuploidy as shown by cytogenetic analysis of interphase nuclei. Nuclear DNA is stained with DAPI (blue), and near-centromeric probes for linkage groups 2 (red) and 16 (green). FIG. 4D shows that BRAF^{V599E} induced tumors show dramatic activation of ERK. Normal (left) and tumor (right) tissue within the liver of a transplanted fish were stained with anti-phospho-ERK (brown stain; 100×), FIG. 4E shows a 400× magnification of the section of normal liver, and FIG. 4F shows same staining of a tumor nodule within the liver displaying high levels of anti-phospho-ERK staining.

### DETAILED DESCRIPTION OF INVENTION

The present invention is directed to a transgenic zebrafish that express activated BRAF specifically in melanocytes and its use in screening for agents that can be used to treat melanomas. The invention is based upon our findings that activated mutant human BRAF, but not wild type BRAF, was able to induce highly visible, ectopic nevi (also known as moles) in a transgenic zebrafish.

Similar to other vertebrates, zebrafish have melanocytes, the black pigmented cells carrying melanin that are derived from the neural crest (Mellgren & Johnson, 2002; Rawls et al., 2001). The microphthalmia transcription factor gene (Mitf) is a critical regulator of melanocyte development, and zebrafish Mitfa is expressed in melanocytes and the retinal pigment epithelium (Lister et al., 1999). Mitf is mutated in the mouse microphthalmia mutant and the zebrafish nacre mutant, both of which lack melanocytes (Widlund & Fisher, 2003; Hodgkinson et al., 1993; Lister et al., 1999). Thus, the melanocyte differentiation program is evolutionarily conserved in the vertebrates (Mellgren & Johnson, 2002).

We have now shown that transgenic expression in zebrafish of mutant, but not wild type, human BRAF under the control of the melanocyte specific Mitfa promoter led to patches of ectopic proliferating melanocytes that resemble nevi (fish “f”-nevi). We have further shown that in a p53-deficient fish mutant, activated BRAF induced formation of f-nevi which rapidly developed into invasive melanomas. The melanomas closely resembled human melanomas and could be serially transplanted. These data provide direct evidence that human BRAF functions in the proliferation of melanocytes and their precursors, in vivo, and that the p53 and BRAF pathways interact genetically to produce melanoma. These lines of transgenic zebrafish provide a unique tool for screening for genetic or chemical modifiers of melanoma.

The term “agent” or “compound” as used herein and throughout the specification means any organic or inorganic molecule, including modified and unmodified nucleic acids such as antisense nucleic acids, RNAi, such as siRNA or shRNA, peptides, peptidomimetics, receptors, ligands, and antibodies.

The term “physical condition” as used herein refers to conditions such as radiation, including ionic or non-ionic radiation, for example, UV radiation, sunlight, or alpha, beta and gamma radiation and other known forms of radiation.

In the methods of the present invention, a variety of test compounds and physical conditions from various sources can be screened for the ability of the compound to alter the melanoma or nevus phenotype or to test the effectiveness of a compound believed to be useful in treating a disease. Compounds to be screened can be naturally occurring or synthetic molecules. Compounds to be screened can also be obtained from natural sources, such as, marine microorganisms, algae, plants, and fungi. The test compounds can also be minerals or oligo agents. Alternatively, test compounds can be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds can include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates, chimeric molecules, known or suspected carcinogens, known or suspected tumor-promoting compounds, radio-protective compounds, radio-sensitizing compounds, free-radical scavenging compounds, free-radical generating compounds, UV-protective compounds, UV-sensitizing compounds, and combinations thereof.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. In the method of the present invention, the preferred test compound is a small molecule, nucleic acid and modified nucleic acids, peptide, peptidomimetic, protein, glycoprotein, carbohydrate, lipid, or glycolipid. Preferably, the nucleic acid is DNA or RNA.

Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the DIVERSet E library (16,320 compounds) from ChemBridge Corporation (San Diego, Calif.), the National Cancer Institute's (NCI) Natural Product Repository, Bethesda, Md., the NCI Open Synthetic Compound Collection, Bethesda, Md., NCI's Developmental Therapeutics Program, or the like.

The compounds may be administered to the zebrafish, for example, by diluting the compounds into the water wherein zebrafish are maintained, mixing the compounds with the zebrafish feed, topically administering the compound in a pharmaceutically acceptable carrier on the fish, using three-dimensional substrates soaked with the test compound such as slow release beads and the like and embedding such substrates into the fish.

Physical exposure to irradiation, UV-irradiation, ionizing radiation, sunlight and other radiation sources is also considered an “agent” as used throughout the specification. Methods of exposing fish to UV light have been described, for example, in Setlow R B, Woodhead A D. “Three unique experimental fish stories: Poecilia (the Past), Xiphophorus (the Present), and Medaka (the Future).” Mar Biotechnol (NY). 2001 June; 3(Supplement 1):S17-23; and Setlow R B, Woodhead A D, Grist E. “Animal model for ultraviolet radiation-induced melanoma: platyfish-swordtail hybrid.” Proc Natl Acad Sci U S A. 1989 November; 86(22):8922-6. Such and similar methods can be used in exposing the fish of the invention to radiation.

The compound formulations may conveniently be presented in unit dosage form, e.g., tablets and sustained release capsules, and in liposomes, and may be prepared by any methods well know in the art of pharmacy. (See, for example, Remington: The Science and Practice of Pharmacy by Alfonso R. Gelmaro (Ed.) 20th edition, Dec. 15, 2000, Lippincott, Williams & Wilkins; ISBN: 0683306472.).

The term “mutation” includes substitution, deletions, inversions, insertions, premature terminations and the like. In one embodiment, the mutation is selected from the group of VAL599GLU (also named as VAL600GLU) (Davies et al. Nature. Jun. 27, 2002; 417(6892):949-54), Mutations of the BRAF gene in human cancer), ARG461ILE, ILE462SER, GLY463GLU, and LYS600GLU (Rajagopalan, H., et al. (Letter) Nature 418: 934, 2002), GLY465VAL and LEU596ARG (Naoki, K., et al., Cancer Res. 62: 7001-7003, 2002), and GLY468ARG, GLY468ALA and ASP593GLY (Lee, J. W., et al., Brit. J Cancer 89: 1958-1960, 2003). Particularly useful BRAF mutations include mutations in its C-terminal kinase domain, such as the T to A transversion at nucleotide 1796 in exon 15 resulting in amino acid substitution V599E (also referred to as nucleic acid mutation T1799A resulting in V600E amino acid change) in the human BRAF gene.

The terms “mole”, “nevus” and “nevi” are used interchangeably throughout the specification and are used to generally describe melanocytic nevi. Benign melanocytic tumors (melanocytic nevi) and malignant tumors (malignant melanoma, melanoma, melanoblastoma) are tumors of melanocytes, cells of neuroectodermal origin. These cells enter the epidermal basal layer during embryonal development. Later produce melanin, brown pigment, which is taken up by surrounding melanocytes. Melanin stains the epidermis brown and protects the body against UV radiation. In normal skin the melanocytes are regularly dispersed within the basal layer of the epidermis. Their cytoplasm is pale and nuclei have fine chromatin. Nevi are generally located anywhere on the body, some are present at birth, most of them appear during childhood and puberty. The present themselves as macules, papules of different shades of brown, sometimes pink or dark blue, the surface is smooth, some lesions are pedunculated, some with hairy so called dysplastic nevi are of irregular borders, variable color (even red) and can be multiple. The common melanocytic nevi as described throughout the specification include, but are not limited to lentigo simplex, junctional melanocytic nevi, compound melanocytic nevi, dermal nevi, and speckled lentiginous nevi (nevus spilus), balloon cell nevus, halo nevus, recurrent melanocytic nevus, giant melanocytic nevus, nevus Spitz, pigmented spindle cell nevus (Reed), Blue nevus, including blue nevus of common type of dendritic melanocytes, cellular blue nevus, special forms of blue nevi, and combined nevi and melanocytic nevus of the conjunctiva, which definitions are well known to one skilled in the art (for examples, see, e.g., Atlas of Dermatology, Melanocytic Tumors at http://atlases.muni.cz/atl en/mail+nadory+melantum.html).

Melanocytes are specialized cells residing in the hair follicles, the eye, and the basal layer of the human epidermis whose primary function is the production of the pigment melanin, giving rise to skin, hair, and eye color. Melanogenesis is a process unique to melanocytes that involves the processing of tyrosine by a number of melanocyte-specific enzymes, including tyrosinase and tyrosinase-related protein 1 (TRP-1). The “melanocyte specific promoter” as used herein and throughout the specification is intended to cover any and all promoters capable of directing melanocyte-specific gene expression. Such promoters include, but are not limited to, for example, tyrosinase promoter (see, e.g., Lowings P., et al. Mol Cell Biol. 1992 August; 12(8): 3653-62); dopachrome tautomerase promoter (see, e.g., Ludwig et al., FEBS Letters Volume 556, Issue 1-3, pp. 236-244, 2003); and melanocyte inducing factor promoter (Mitf) (see, e.g., Shibahara et al., Pigment Cell Research, Volume 13 Issue Supplement 8 Page 98—June 2000). In one preferred embodiment, the melanocyte specific promoter is Mitfa-promoter.

The term “proto-oncogene” and “tumor suppressor gene” as used in the specification are meant to cover, for example, the following tumor suppressor genes and/or proto-oncogenes. The GenBank Accession No. for at least one nucleic acid encoding the named gene is shown in parenthesis after the name of the gene. Proto-oncogenes and/or tumor suppressor genes useful according to the present invention include, but are not limited to isolated and purified p53 (M14694; M14695), myc proto-oncogene (V00568), erbB3 (M29366), CSF1R (X03663), MDM2 (Z12020, M92424), sis proto-oncogene (X02811; X02744; M12783; M16288), myb proto-oncogene (M15024), c-kit proto-oncogene (X06182), THRA1 or v-erbA-related protein ear-1 (M24898), erbB proto-oncogene (X00588; K031193; X00663; U48722), EAR2 (X12794), EAR3 (X12795; X16155; X58241), TEL proto-oncogene (U11732), erbB4 (L07868), jun proto-oncogene (J04111), erbB2 proto-oncogene (M95667; M11730), UFO (M76125), TYRO3 (D17517), MERLIN or NF2 (L11353; Z22664; X72657; L27133) ACK1 (L13738), mas (M13150), pim-1 (M54915), NME2 (L16785; M36981), NF1 (M60915), MCC (M62397), LUCA1 (U03056), ski oncogene (X15218), snoN oncogene (X15219), WT1 (X51630), prohibitin (S85655; U17179), raf1 proto-oncogene (X03484), ab1 proto-oncogene (M14752), src proto-oncogene (K03214; X03996), re1 proto-oncogene (X75042), RHOA (L25080), DCC (X76132), met proto-oncogene (J02958), ABL2 (M35296), KRAS2 (M54968), c-fos proto-oncogene (K00650), NOTCH2 (U77493), int3 proto-oncogene (U95299), prefoldin 5 (D89667), DMDX (AF007111), p33ING1 (AF001954), RBL2 (X74594), notch homolog (M99437), notch homolog 1 (M73980), jun-B (M29039), 5T4 oncofetal trophoblast glycoprotein (Z29083), neogenin (U61262), MAD homolog 1 (U57456), CBL-B (U26710), EB1 protein (U24166), p57 KIP2 (U22398), ETS oncogene (L16464), TROB transducer of erbB2.1 (D38305), JunD (X56681), ezrin (X51521), FOS-related antigen 1 (X16707), FOS-related antigen 2 (X16706), N-ras proto-oncogene (X02751), BRCA2 (U43746), cyclin-dependent kinase 4 inhibitor 2D (U40343; U20498), APC (M74088; M73548), c-fgr proto-oncogene (M19722), L-myc proto-oncogene (M19720), YES1 (M15990), RB1 (M15400), N-myc proto-oncogene (M13228), and PTEN (GeneBank Accession Nos. U92436, U96180).

Other useful cancer related genes, many of which are proto-oncogenes and/or tumor suppressor genes are disclosed in Futreal et al. A Census of Human Cancer Genes, Nature Reviews, 4: 177-183, 2004, and on the Sanger C4enter web site at www.sanger.ac.uk.

The term “adult fish” as used herein and throughout the specification refers to the fish that exhibit the phenotype of nevi or melanoma. Changes at the melanocyte level can be seen as early as 3 weeks of development.

The phrase “regression of size” is based in the visual detection of the nevi and melanomas. Measurement can be done using photography or video screening system.

The phrase “genetically modified fish” as used in the specification refers to zebrafish that expresses a human BRAF in its melanocytes. Expression can be either transient or stable. The zebrafish are genetically modified using methods well known to one skilled in the art. Detailed methods to grow and manipulate zebrafish are available, for example at ZFIN web site at www.zfin.org (THE ZEBRAFISH BOOK A guide for the laboratory use of zebrafish Danio (Brachydanio) rerio by Monte Westerfield, Institute of Neuroscience, University of Oregon).

Direct nucleic acid injection of the expression constructs comprising an appropriate promoter, such as a mitfa-promoter, sequence operably linked to the gene of interest such as a mutant BRAF encoding nucleic acid or tumor suppressor or proto-oncogene encoding nucleic acid. Methods described in Fan L., et al. using cell-mediated gene transfer and targeted mutagenesis using pluripotent zebrafish embryonic stem (ES) cells can also be used to generate the zebrafish according to the present invention (Methods Mol Biol. 2004; 254: 289-300). Further, method described by Kurita K., et al., can be used to create transgenic fish using sperm genetically modified and grown in a laboratory dish (Proc Natl Acad Sci U S A. Feb. 3, 2004; 101(5):1263-7. Epub Jan. 26, 2004). Kurita et al. describe a method for production of transgenic zebrafish from cultured sperm. The sperm were differentiated from premeiotic germ cells infected with a pseudotyped retrovirus in vitro. Similar method can be used to prepare the zebrafish according to the present invention.

The benign nevi and malignant melanoma can be distinguished histologically. For example, in situ malignant melanoma (malignant melanocytes scattered in all epidermal layers) show atrophic epidermis, prominent dermal solar elastosis and almost always lymphocytic infiltration. Invasion of the dermis by melanocytes may occur in lentigo maligna melanoma.

Other methods that can be used to detect melanoma include, but are not limited to immunohistochemistry using the melanoma specific antibody HMB-45, or RT-PCR with different melanoma associated antigens (MAA) including, but not limited to tyrosinase, MART-1/Melan A, Pmel-17, TRP-1, and TRP-2 (see, e.g., Hatta N., et al., J Clin Pathol. 1998 August; 51(8): 597-601).

In an effort to determine the effect on melanocyte development of activated BRAF, we utilized the Mitfa promoter to drive expression of BRAF in zebrafish embryos. One-cell stage zebrafish embryos were microinjected with mitfa-BRAF or mitfa-BRAF^{V599E}, the most common mutation associated with human nevi. BRAF^{V599E} injected zebrafish pigment patterns were not altered in early embryos, but ectopic melanocyte pigmentation patterns could be seen as early as about week 3 of development.

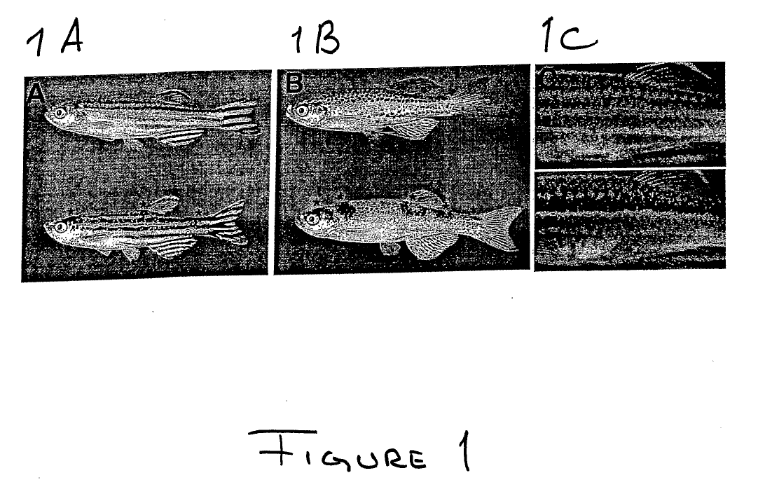

By about 8 weeks, the melanocytes proliferations were clearly evident. Of 372 injected fish, 41 ad ectopic black melanocytic f-nevi (11.02%; Table 1). The number and size of f-nevi varied among fish, ranging from a few melanocytes clustered in a discrete spot to pigmention that covered large areas (some over 40%) of the surface of the fish (FIG. 1). The ectopic melanocyte proliferations were seen in the wild type striped backgrounds (AB, and Tubingen backgrounds; Table 1), but most easily seen in the leopard background. Fish were monitored closely over time, and generally fish that did not have f-nevi by four months of age did not acquire them at a later time point. Some fish with f-nevi did infrequently continue to acquire more spots. Our results show that the most common BRAF mutation in humans is clearly capable of inducing a dramatic change in pigmentation patterns, consistent with a probable BRAF role in human nevi development (Pollock et al., 2003).

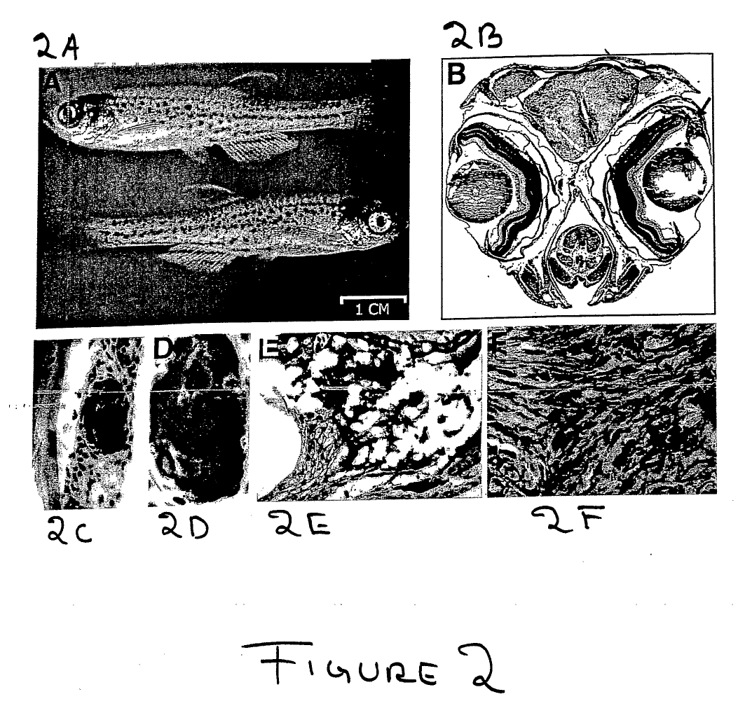

Histological examination of f-nevi revealed a range of excess melanocytes and melanin in the dermis and eye of the fish (FIG. 2A, FIG. S1, A). Although there is an expansion of melanocytes, f-nevi do not directly resemble human nevi. Zebrafish melanocytes in f-nevi exhibit more abundant dendritic cytoplasm compared to small round cells with minimal cytoplasm in human melanocytes in nevi. While mitfa-BRAF^{V599E} induced the expansion of melanocytes, the melanocytes did not appear neoplastic. Without wishing to be bound by theory, these data indicate that nacre-BRAF^{V599E} is required for the formation of f-nevi, and additional mutations are required during the progression to melanoma. This is consistent with analysis of benign and dysplastic nevi, as well as primary melanomas showing BRAF^{V599E} mutations. Given the importance of BRAF in nevus formation in humans (Pollock et al., 2003), we believe that these BRAF driven melanocytic proliferations in zebrafish are the biological equivalent of a human nevus.

To study the biology of BRAF^{V599E} expressed in all melanocytes we generated stable transgenic nitfia-BRAF^{V599E} zebrafish. Surprisingly, we identified two transgenic lines that produced offspring that had an overt phenotype of extra melanocytes, particularly in the dorsal axis, and interfering with the most dorsal stripe patterning (FIG. 1C). The width of each stripe was larger than wild type fish. On the leopard background, each of the spots was larger, containing more melanocytes, while the general pattern of melanocytes was not disrupted. The general expansion of melanocytes in the formation into f-nevi is more prominent in the transient transgenic animal, potentially due to differences in the level of BRAF expression. The stable transgenic fish demonstrate the disruption of normal melanocytes by BRAF^{V599E}, consistent with the transient transgenic analysis.

Therefore, in one embodiment, the present invention provides a transgenic zebrafish that is useful as a model to screen agents to treat nevi, the fish expressing a transgene, wherein a melanocyte specific promoter drives the expression of mutant BRAF (NCBI Protein sequence ID NO. P15056; nucleic acid sequence encoding BRAF gi:1170701). In one preferred embodiment, the BRAF has one or more mutations in its kinase domain comprising amino acids from 456 . . . 716 in P15056 protein sequence. In one preferred embodiment, the expression construct is mitfa-BRAF^{V599E}, comprising a mitfa-promoter and BRAF mutant with a substitution V599E.

Without wishing to be bound by a theory, we hypothesized that BRAF activation was an early event in nevi formation, and that additional genetic defects would be necessary for the progression to malignant melanoma.

It is known that mutations in p53 are surprisingly low in melanomas, although germline p14^{ARF} mutations and MDM2 activation have been hypothesized to inactivate the p53 pathway in the genesis of melanomas (Randerson-Moor et al., 2001; Rizos et al., 2001; Polsky et al., 2001; Gelsleichter et al., 1995; Poremba et al., 1995). The conspicuous lack of p53 mutations in RAS induced Ink4a/Arf-/-melanomas in mice, in conjunction with other evidence, suggested that the p53 pathway may suppress RAS-induced melanoma formation (Chin et al., 1997; Sharpless & Chin 2003). Substantiating this, p53 mutations have been shown to cooperate with activated RAS in the generation of amelanotic melanomas in mice (Kannan et al., 2003; Sharpless & Chin, 2003; Sharpless et al., 2002; Bardeesy et al., 2001). To test if p53 deficiency promotes the formation of melanoma from f-nevi in zebrafish generated by activated BRAF, we injected mitfa-BRAF^{V599E} into zebrafish embryos harboring a homozygous exon 7 missense mutation (MET214LYS) (nucleotides 6633-6816 of GenBank ID No. gi:42406304) mutation in the TP53 gene (Bergqvist Anticancer Res. 2003 March-April; 23(2B): 1207-12. The p53 Met214Lys mutation is found in 7/103 human cancers (IARC TP53 mutation database version R8, June 2003, at http://www.iarc.fr/p53/, Olivier M, Eeles R, Hollstein M, Khan M A, Harris C C, Hainaut P. The IARC TP53 Database: new online mutation analysis and recommendations to users. Hum Mutat. 2002 June; 19(6): 607-14). Checkpoint deficient, heterozygous fish do not initiate apoptosis after irradiation, and homozygous p53 develop neural tumors at 11 months. Our results showed that 9 out of 66 fish (13.6%) injected embryos developed f-nevi, and a subset of these animals developed malignant melanoma.

Therefore, in one embodiment, the invention provides a zebrafish melanoma model wherein the fish expresses a combination of a tumor suppressor gene operably linked to a promoter and a mutant human BRAF gene operably linked to a melanocyte-specific promoter. In one preferred embodiment, the tumor suppressor gene is a mutant p53 and the melanocyte specific promoter is mitfa-promoter.

In one embodiment, the invention provides methods to identify compounds capable of inhibiting melanoma growth. The method comprises administering a test compound or a mixture of test compounds to the transgenic zebrafish, in which the transition to melanoma occurs relatively rapidly, for example, within about a ten-day period.

According to the method of the present invention, the transgenic fish or fish population are administered one or more test compounds either alone or in combination and the appearance of the melanomas is observed. The tumor/nevus lesions are evaluated by the size, histology, immunohistochemistry and/or mRNA or protein expression using RT-PCR or Western blot analysis of melanoma specific and/or melanocyte proteins. The effectiveness of the test compounds is determined by comparing development of melanomas/nevi in fish that have been treated with the test compound to those fish with the same genetic makeup that have not been treated with the test compounds.

Typically, a non-treated f-nevi become white in appearance, and there is an increase in size of the lesion. Therefore, if the lesion in the treated fish does not increase in size or increases less or slower than in the non-treated fish or fish population the test compound is deemed to have an effect in inhibiting melanoma growth and/or formation of nevi and/or conversion of nevi to melanoma. In addition, the fish tumor becomes more pigmented over an about ten day period. Therefore, if the pigmentation in the treated fish progresses more slowly or fails to occur, the test compound is deemed to have an effect in inhibiting melanoma growth and/or formation of nevi and/or conversion of nevi to melanoma. Also, the histological examination of the fish melanomas shows a poorly differentiated, pigmented, highly aggressive and invasive melanoma with nuclear pleomorphism, with similarities to melanoma in humans. Therefore, if the histological features include better or normal differentiation, pigmentation and less aggressive and/or invasive melanocytes with no nuclear pleorphisms, the test compound is considered to have an effect in inhibiting melanoma growth and/or formation of nevi and/or conversion of nevi to melanoma.

Western blot analysis of fish tumor extracts confirmed the expression of the myc-tagged BRAF^{V599E} transgene. Spontaneous melanomas are exceedingly rare in zebrafish, and examination of over 10,000 DMBA treated zebrafish failed to identify a single melanoma. Therefore, the zebrafish melanoma model of the present invention provides strong evidence for the interaction of the BRAF and p53 pathways in melanoma development.

Raf kinases participate in MAP kinase signaling, functioning as a MAP kinase kinase kinase (MAPKKK), and MAP kinase signaling is important for melanocyte proliferation. (Satyamoorthy et al., 2003; Halaban, R. 2002; Busca et al., 2000).

Mutational activation of N-ras has been shown to occur in a subset of melanoma, and ras is an upstream activator of the MAP kinase pathway. To analyze MAP kinase pathway in f-nevi and melanoma, we performed immunohistologic analysis using anti-Erk and anti-phospho-Erk antibodies (Carr et al., Gene-expression profiling in human cutaneous melanoma, Oncogene. 2003 May 19; 22(20): 3076-80).

Activation of the Ras pathway coupled with loss of the INK4a/ARF locus are signature genetic events in melanoma development. In the activated RAS melanoma model, p16^{INK4a-l-} mice acquire somatic p53 pathway lesions, and conversely, p19^{ard-l-} mice lose p16^{INK4a} function (Kannan et al., 2003; Sharpless & Chin, 2003; Sharpless et al. 2003; Bardessy et al., 2001).

Therefore, in one embodiment, the present invention provides somatic mutations that are acquired in the BRAF+p53−/− fish. For example, melanomas in the RAS+ p53−/− mice overexpress myc, which, without wishing to be bound by a particular theory, may serve as an Rb-pathway lesion (Bardessy et al., 2001).

The melanomas generated by activated BRAF and p53 deficiency in fish are pigmented, in contrast to the RAS induced melanomas in mice (Chin et al., 1997; Sharpless & Chin, 2003). This may reflect species differences in the generation of melanoma. In this regard, the fish appears to more closely approximate the human disease since melanomas are often pigmented. It is also possible that RAS and BRAF activate overlapping, and perhaps epistatic genetic signaling pathways that enhance or inhibit differentiation, in addition to providing a strong proliferation signal to melanoblasts. For instance, some of the signals are likely the BRAF activation of MAP kinase pathway downstream of RAS. Again, without wishing to be bound by a theory, we suggest that BRAF activation is required for the initiation of melanoma development, and that other deficiencies, such as loss of p53 pathway function, are required for the progression to metastatic disease.

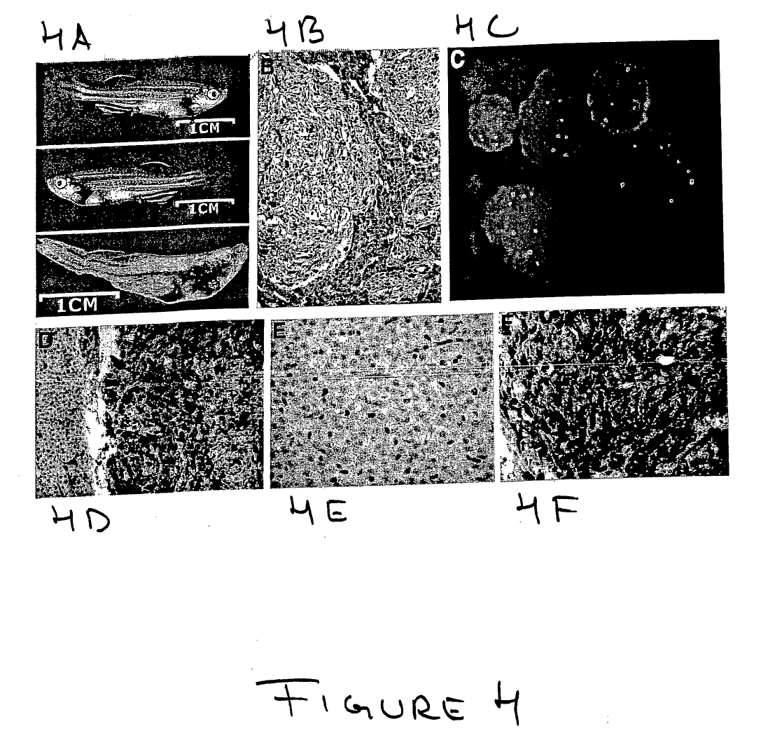

In addition, a characteristic of malignant melanoma is their transplantability. A portion of the melanoma was transplanted intraperitoneally into seven gamma irradiated wild type adult zebrafish. Sub-lethal irradiation with 20 Gy allows transplantation between immunologically heterologous zebrafish (Langenau et al., 2003; Traver et al., 2003). Black tissue was visible at the site of injection within about 2 weeks after injection, and melanoma was apparent through the body of the adult fish by about 3 weeks after injection. Sectioning of the injected fish revealed aggressive melanoma disease invading multiple structures, including the gut lamina propria, heart, liver, pancreas, kidney marrow and possibly the blood stream (FIG. 4B, S3A). All seven adults injected with melanoma succumbed to disease, in contrast to those injected with saline solution alone. These experiments establish the transplantability of the zebrafish melanomas, confirming the BRAF^{V599E}-p53 induced tumors have genuine malignant properties.

Therefore, the determination of the capacity of a test compound to inhibit melanoma growth may also be determined by transplanting the developing melanomas from the treated and non-treated fish to, for example, gamma irradiated wild type adult zebrafish. If black tissue does not become visible at the site of injection wherein the treated fish cells are used within about 1-3 weeks, preferably about 2 weeks after injection, and melanoma is not apparent through the body of the adult fish by about 2-4 weeks, preferably about 3 weeks after injection the compound is considered to have an effect in inhibiting the melanoma growth. Sectioning of the injected fish revealed aggressive melanoma disease invading multiple structures, including the gut lamina propria, heart, liver, pancreas, kidney marrow and possibly the blood stream. Therefore, one method to determine the effectiveness of the test agent is to section the fish that have received transplanted cells. After sectioning the fish that have received transplanted cells from the treated and non-treated fish if fewer or no organs are affected with malignant growth in the fish that have received transplanted cells from the treated fish compared to the non-treated fish, then the compound is considered to have an effect in inhibiting melanoma growth.

Whereas the vast majority of melanomas show chromosome abnormalities and genetic instability, benign nevi mostly do not show such abnormalities. We examined the cytogenetics of the f-nevi, and melanomas (Bastian B. C., et al., Classifying melanocytic tumors based on DNA copy number changes. Am J Pathol. 2003 November; 163(5): 1765-70.).

Melanoma is an epidemic cancer, notoriously aggressive and unresponsive to therapy. The zebrafish model that has been established here has significant potential for dissecting the molecular pathways that are altered during melanoma production and potentially can be used to define new therapies. Expression of the most common mutation in melanomas and nevi, BRAF^{V599E}, is highly efficient at promoting melanocyte proliferation. The f-nevi alone are not neoplastic, but become highly aggressive and invasive melanomas when compromised for the p53 pathway. With a large number of cell cycle and tumor suppressor mutants being recently available in the zebrafish field (Shepard et al., 2004; Amatruda et al., 2002; Stem & Zon, 2003; J. Amatruda, J. Shepard, K. Phaff, E. E. Patton, C. Straub, & L. I. Zon, unpublished data), this type of genetic interaction for cancer can be easily explored in the zebrafish system. This system complements other genetic systems such as Xiphophorus and mouse that are being used to study melanoma (Walter & Kazianis, 2001; Hjappan et al., 2003). The advantage of the zebrafish model lies in the facile methods to undertake genetic and chemical screens for suppression or enhancement of phenotypes (Patton et al., 2001; MacRae & Peterson, 2003). The exceptional visibility of the tumors, and the ability to directly follow the progression of the tumor from nevi to metastatic melanoma will facilitate the biology. Etiological risk such as sun exposure, coupled with genetic factors for melanoma, can be explored.

It would also be useful to identify compounds that affect expression of the mutant genes that play a role in the development of melanoma. The disclosed transgenic fish can be exposed to compounds to assess the effect of the compound on the expression of a gene of interest, such as the tumor suppressor gene or the mutant BRAF gene. For example, test compounds can be administered to transgenic fish harboring the mutant BRAF and/or tumor suppressor gene operably linked to a reported gene, such as a green fluorescent protein (GFP) encoding gene. By comparing the expression of the reporter protein in fish exposed to a test compound to those that are not exposed, the effect of the compound on the expression of the mutant BRAF and/or tumor suppressor gene can be assessed.

The fish of the present invention can also be used in genetic screenings to identify fish genes that participate in regulation of tumor formation induced by mutant BRAF. Such genetic screens are well documented for zebrafish.

### EXAMPLES

In an effort to determine the effect on melanocyte development of activated BRAF, we utilized the Mitfa promoter to drive expression of BRAF in zebrafish embryos. One-cell stage zebrafish embryos were microinjected with mitfa-BRAF or mitfa-BRAF^{V599E}, the most common mutation associated with human nevi. BRAF^{V599E} injected zebrafish pigment patterns were not altered in early embryos, but ectopic melanocyte pigmentation patterns could be seen as early as week 3 of development. By 8 weeks, the melanocytes proliferations were clearly evident. Of 372 injected fish, 41 had ectopic black melanocytic f-nevi (11.02%; Table 1). The number and size of f-nevi varied among fish, ranging from a few melanocytes clustered in a discrete spot to pigmention that covered large areas (some over 40%) of the surface of the fish (FIG. 1). The ectopic melanocyte proliferations were seen in the wild type striped backgrounds (AB, and Tubingen backgrounds; Table 1), but most easily seen in the leopard background. Fish were monitored closely over time, and generally fish that did not have f-nevi by four months of age did not acquire them at a later time point. Some fish with f-nevi did infrequently continue to acquire more spots. Our results show that the most common BRAF mutation in humans is clearly capable of inducing a dramatic change in pigmentation patterns, consistent with a probable BRAF role in human nevi development (Pollock et al., 2003).

Histological examination of f-nevi revealed a range of excess melanocytes and melanin in the dermis and eye of the fish (FIG. 2A, FIG. S1, A). Although there is an expansion of melanocytes, f-nevi do not directly resemble human nevi. Zebrafish melanocytes in f-nevi exhibit more abundant dendritic cytoplasm compared to small round cells with minimal cytoplasm in human melanocytes in nevi. While mitfa-BRAF^{V599E} induced the expansion of melanocytes, the melanocytes did not appear neoplastic. These data indicate that nacre-BRAF^{V599E} is required for the formation of f-nevi, and additional mutations are required during the progression to melanoma. This is consistent with analysis of benign and dysplastic nevi, as well as primary melanomas showing BRAF^{V599E} mutations. Given the importance of BRAF in nevus formation in humans (Pollock et al., 2003), we believe that these BRAF driven melanocytic proliferations in zebrafish are the biological equivalent of a human nevus.

To study the biology of BRAF^{V599E} expressed in all melanocytes we generated stable transgenic mitfa-BRAF^{V599E} zebrafish. Two transgenic lines produced offspring that had an overt phenotype of extra melanocytes, particularly in the dorsal axis, and interfering with the most dorsal stripe patterning (FIG. 1C). The width of each stripe was larger than wild type fish. On the leopard background, each of the spots was larger, containing more melanocytes, while the general pattern of melanocytes was not disrupted The general expansion of melanocytes in the formation into f-nevi is more prominent in the transient transgenic animal, potentially due to differences in the level of BRAF expression. The stable transgenic fish demonstrate the disruption of normal melanocytes by BRAF^{V599E}, consistent with the transient transgenic analysis.

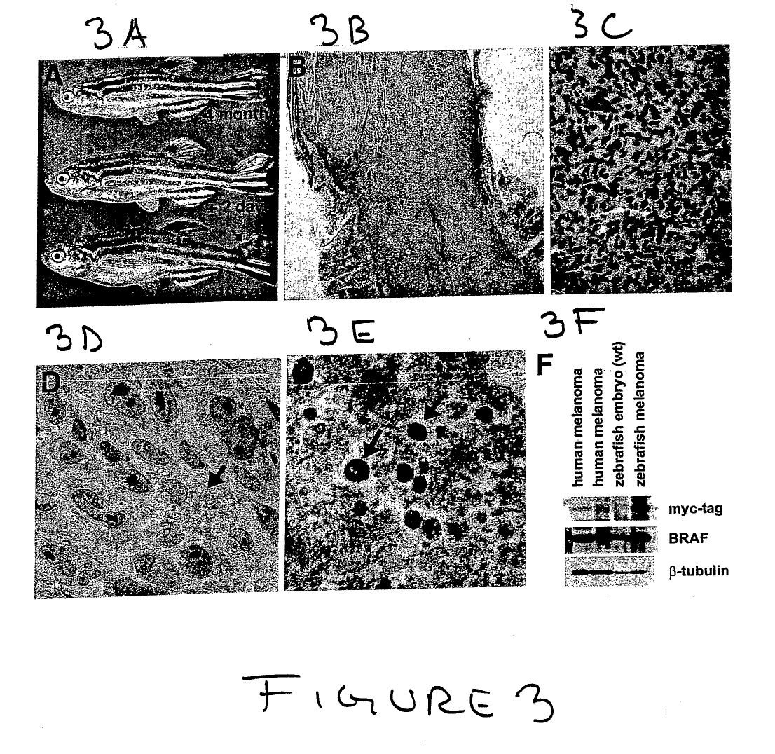

We hypothesized that BRAF activation was an early event in nevi formation, and that additional genetic defects would be necessary for the progression to malignant melanoma. Mutations in p53 are surprising low in melanomas, although germline p14^{ARF} mutations and MDM2 activation have been hypothesized to inactivate the p53 pathway in the genesis of melanomas (Randerson-Moor et al., 2001; Rizos et al., 2001; Polsky et al., 2001; Gelsleichter et al., 1995; Poremba et al., 1995). The conspicuous lack of p53 mutations in RAS induced Ink4a/Arf-/- melanomas in mice, in conjunction with other evidence, suggested that the p53 pathway can suppress RAS-induced melanoma formation (Chin et al., 1997; Sharpless & Chin 2003). Substantiating this, p53 mutations have been shown to cooperate with activated RAS in the generation of amelanotic melanomas in mice (Kannan et al., 2003; Sharpless & Chin, 2003; Sharpless et al., 2002; Bardeesy et al., 2001). To test if p53 deficiency promotes the formation of melanoma from f-nevi in zebrafish generated by activated BRAF, we injected mitfa-BRAF^{V599E} in zebrafish embryos harboring a homozygous exon 7 mutation in the TP53 gene (Bergmans, Murphy et al., 2004). The p53 Met214Lys mutation is found in 7/103 human cancers. Checkpoint deficient, heterozygous fish do not initiate apoptosis after irradiation, and homozygous p53 develop (neural) tumors at 11 months. 9 out of 66 fish (13.6%) injected embryos developed f-nevi, and a subset of these animals developed malignant melanoma. The transition to melanoma occurred rapidly within a ten-day period (FIG. 3A). Typically, an f-nevi becomes white in appearance, and there is an increase in size of the lesion. The tumor becomes more pigmented over a ten day period. Histological examination revealed this to be a poorly differentiated, pigmented, highly aggressive and invasive melanoma with nuclear pleomorphism, with similarities to melanoma in humans (FIG. 3B and C). Western blot analysis of tumor extracts confirmed the expression of the myc-tagged BRAF^{V599E} transgene (FIG. 3D). Spontaneous melanomas are exceedingly rare in zebrafish, and examination of over 10,000 DMBA treated zebrafish failed to identify a single melanoma (Amatruda, Shepard, Stem, Murphy, Belair & Zon, unpublished data). This zebrafish melanoma model provides strong evidence for the interaction of the BRAF and p53 pathways in melanoma development.

Raf kinases participate in MAP kinase signaling, functioning as a MAP kinase kinase kinase (MAPKKK), and MAP kinase signaling is important for melanocyte proliferation. (Satyamoorthy et al., 2003; Halaban, R. 2002; Busca et al., 2000). Mutational activation of N-ras has been shown to occur in a subset of melanoma, and ras is an upstream activator of the MAP kinase pathway. To analyze MAP kinase pathway in f-nevi and melanoma, we performed immunohistolgical analysis using anti-Erk and anti-phospho-Erk antibodies.

Activation of the Ras pathway coupled with loss of the INK4a/ARF locus are signature genetic events in melanoma development. In the activated RAS melanoma model, p16^{INK4a-l-} mice acquire somatic p53 pathway lesions, and conversely, p19^{Ard-/-} mice lose p16^{INK4a} function (Kannan et al., 2003; Sharpless & Chin, 2003; Sharpless et al. 2003; Bardessy et al., 2001). It is our future interest to explore somatic mutations that may be acquired in our BRAF+p53-/- fish. For example, melanomas in the RAS+ p53-/- mice overexpress myc, which may serve as an Rb-pathway lesion (Bardessy et al., 2001). The melanomas generated by activated BRAF and p53 deficiency in fish are pigmented, in contrast to the RAS induced melanomas in mice (Chin et al., 1997; Sharpless & Chin, 2003). This may reflect species differences in the generation of melanoma. In this regard, the fish appears to more closely approximate the human disease since melanomas are often pigmented. It is also possible that RAS and BRAF activate overlapping, and perhaps epistatic genetic signaling pathways that enhance or inhibit differentiation, in addition to providing a strong proliferation signal to melanoblasts. For instance, some of the signals are likely the BRAF activation of MAP kinase pathway downstream of RAS. We suggest that BRAF activation is required for the initiation of melanoma development, and that other deficiencies, such as loss of p53 pathway function, are required for the progression to metastatic disease.

A characteristic of malignant melanoma is their transplantability. A portion of the melanoma was transplanted intraperitoneally into seven gamma irradiated wild type adult zebrafish. Sub-lethal irradiation with 20 Gy allows transplantation between immunologically heterologous zebrafish (Langenau et al., 2003; Traver et al., 2003). Black tissue was visible at the site of injection within 2 weeks after injection, and melanoma was apparent through the body of the adult fish by 3 weeks after injection (FIG. 4A). Sectioning of the injected fish revealed aggressive melanoma disease invading multiple structures, including the gut lamina propria, heart, liver, pancreas, kidney marrow and possibly the blood stream (FIG. 4B, S3A). All seven adults injected with melanoma succumbed to disease, in contrast to those injected with saline solution alone. These experiments establish the transplantability of the zebrafish melanomas, confirming the BRAF^{V599E}-p53 induced tumors have genuine malignant properties.

Whereas the vast majority of melanomas show chromosome anomalies and genetic instability, benign nevi mostly do not show abnormalities (Bastian et al., 2003). We examined the cytogenetics of the fish-nevi and melanomas.

Melanoma is an epidemic cancer, notoriously aggressive and unresponsive to therapy. The zebrafish model that has been established here has significant potential for dissecting the molecular pathways that are altered during melanoma production and potentially can be used to define new therapies. Expression of the most common mutation in melanomas and nevi, BRAF^{V599E}, is highly efficient at promoting melanocyte proliferation. The f-nevi alone are not neoplastic, but become highly aggressive and invasive melanomas when compromised for the p53 pathway. With a large number of cell cycle and tumor suppressor mutants being recently available in the zebrafish field (Shepard et al., 2004; Amatruda et al., 2002; Stem & Zon, 2003; J. Amatruda, J. Shepard, K. Phaff, E. E. Patton, C. Straub, & L. I. Zon, unpublished data), this type of genetic interaction for cancer can be easily explored in the zebrafish system. This system complements other genetic systems such as Xiphophorus and mouse that are being used to study melanoma (Walter & Kazianis, 2001; Hjappan et al., 2003). The advantage of the zebrafish model lies in the facile methods to undertake genetic and chemical screens for suppression or enhancement of phenotypes (Patton et al., 2001; MacRae & Peterson, 2003). The exceptional visibility of the tumors, and the ability to directly follow the progression of the tumor from nevi to metastatic melanoma will facilitate the biology. Etiological risk such as sun exposure, coupled with genetic factors for melanoma, can be explored.

### REFERENCES

All the references cited herein and throughout the specification are herein incorporated by reference in their entirety.

Amatruda J F, Shepard J L, Stern H M, Zon L I. Cancer Cell. 2002 April; 1(3): 229-31.

Bardeesy et al., Mol. Cell. Biol. 21, 2144-2153 (2001)

Bastian, B C, Oncogene 22, 3081-3086 (2003)

Chin et al., Genes Dev., 11, 2822-2834 (1997)

Davies et al., Nature 417, 949-954 (2002)

Gelsleichter et al., 1995 Mod. Pathol 8: 530-535

Hodgkinson et al., 1993 Mutations at the mouse microphthalmia locus are associated with defects in a gene encoding a novel basic-helix-loop-helix-zipper protein.

Cell. Jul. 30, 1993; 74(2): 395-404.

Jhappan C, Noonan F P, Merlino G. Ultraviolet radiation and cutaneous malignant melanoma. Oncogene. May 19, 2003; 22(20): 3099-112.

Langenau, D. M. et al Science. Feb. 7, 2003; ; 299(5608): 887-90.

Lister J A, Robertson C P, Lepage T, Johnson S L, Raible D W., Development. 1999 September; 126(17): 3757-67.

MacRae C A, Peterson R T. Chem Biol. 2003 October; 10(10): 901-8.

Mellgren E M, Johnson S L. The evolution of morphological complexity in zebrafish stripes. Trends Genet. 2002 March; 18(3): 128-34.

Olivier M, Eeles R, Hollstein M, Khan M A, Harris C C, Hainaut P. The IARC TP53 Database: new online mutation analysis and recommendations to users (version R8). Hum Mutat. 2002 June; 19(6): 607-14.

Patton E E, Zon L I. Nat Rev Genet. 2001 December; 2(12): 956-66.

Pollock et al., Nature Genetics 33, 19-20 (2003).

Polsky D, Bastian B C, Hazan C, Melzer K, Pack J, Houghton A, Busam K, Cordon-Cardo C, Osman I. Ronald 0. Perelman. HDM2 protein overexpression, but not gene amplification, is related to tumorigenesis of cutaneous melanoma. Cancer Res. Oct. 15, 2001; 61(20): 7642-6.

Poremba et al., 1995 Oncol. Res 7: 331-339

Rawls J F, Mellgren E M, Johnson S L. Dev Biol. Dec. 15, 2001; 240(2): 301-14.

Rawls J F, Mellgren E M, Johnson S L. How the zebrafish gets its stripes. Dev Biol. Dec. 15, 2001; 240(2): 301-14.

Sharpless N E, Kannan K, Xu J, Bosenberg M W, Chin L. Both products of the mouse Ink4a/Arf locus suppress melanoma formation in vivo. Oncogene. Aug. 7, 2003; 22(32): 5055-9.

Sharpless, L. Chin, Oncogene 22, 3092-3098 (2003).

Shepard et al., submitted.

Stern H M, Zon L I. Nat Rev Cancer. 2003 July; 3(7): 533-9.

Traver D, Paw B H, Poss K D, Penberthy W T, Lin S, Zon L I. Nat Immunol. 2003 December; 4(12): 1238-46. Epub Nov. 09, 2003.

Widlund H R, Fisher D E. Oncogene. May 19, 2003; 22(20): 3035-41

1. A method for screening for an agent with an ability to regress a mole comprising administering a test agent to a zebrafish, which has been genetically manipulated to express a nucleic acid encoding a mutant human BRAF protein wherein said BRAF encoding protein comprises a mutation in the kinase domain and wherein the mutant human BRAF protein encoding nucleic acid is under a melanocyte-specific promoter thereby resulting in a zebrafish which develops visible nevi as an adult zebrafish, wherein reduction in the size and/or number of the nevi on the zebrafish skin after exposure to the test agent indicates that the test agent has the ability to regress mole formation.

2. A method for screening for an agent with an ability to inhibit melanoma growth comprising administering a test agent to a melanoma model zebrafish, which has been genetically modified to express a nucleic acid encoding a mutant proto-oncogene and a nucleic acid encoding a mutant human BRAF protein wherein said BRAF encoding protein comprises a mutation in the kinase domain and wherein the mutant human BRAF protein encoding nucleic acid is under a melanocyte-specific promoter, said fish developing visible melanomas in an adult fish, wherein reduction of the size and/or the number of the visible melanoma growth and/or inhibition of the melanoma cell proliferation rate and/or regression of the melanoma cells into nevi after exposure to the test agent indicates that the agent has the ability to inhibit melanoma growth.

3. A method for screening for an agent with the ability to inhibit conversion from nevi into melanoma comprising administering a test agent to a melanoma model zebrafish, which has been genetically modified to express a nucleic acid encoding a mutant proto-oncogene and a nucleic acid encoding a mutant human BRAF protein wherein said BRAF encoding protein comprises a mutation in the kinase domain and wherein the mutant human BRAF protein encoding nucleic acid is under a melanocyte-specific promoter said fish developing visible melanomas in an adult fish, wherein inhibition of the conversion from nevi to melanoma is indicative of the test agent having the ability to prevent the nevi from converting to melanoma.

4. A method for screening for a tumor-promoting agent or physical treatment with an ability to promote mole formation comprising administering a test agent to a zebrafish, which has been genetically manipulated to express a nucleic acid encoding a mutant human BRAF protein wherein said BRAF encoding protein comprises a mutation in the kinase domain and wherein the mutant human BRAF protein encoding nucleic acid is under a melanocyte-specific promoter thereby resulting in a zebrafish which develops visible nevi as an adult zebrafish, wherein increase in the size and/or number of the nevi on the zebrafish skin after exposure to the test agent indicates that the test agent has the ability to promote mole formation.

5. A method for screening for a tumor-promoting agent or physical treatment with an ability to promote melanoma growth comprising administering a test agent to a melanoma model zebrafish, which has been genetically modified to express a nucleic acid encoding a mutant proto-oncogene and a nucleic acid encoding a mutant human BRAF protein wherein said BRAF encoding protein comprises a mutation in the kinase domain and wherein the mutant human BRAF protein encoding nucleic acid is under a melanocyte-specific promoter, said fish developing visible melanomas in an adult fish, wherein increase of the size and/or the number of the visible melanoma growth and/or promotion of the melanoma cell proliferation rate and/or progression of the melanoma cells into a more invasive or malignant state after exposure to the test agent indicates that the agent has the ability to promote melanoma growth.

6. A method for screening for a tumor-promoting agent or physical treatment with the ability to promote conversion from nevi into melanoma comprising administering a test agent to a melanoma model zebrafish, which has been genetically modified to express a nucleic acid encoding a mutant proto-oncogene and a nucleic acid encoding a mutant human BRAF protein wherein said BRAF encoding protein comprises a mutation in the kinase domain and wherein the mutant human BRAF protein encoding nucleic acid is under a melanocyte-specific promoter said fish developing visible melanomas in an adult fish, wherein promotion of the conversion from nevi to melanoma is indicative of the test agent having the ability to promote the nevi converting to melanoma.

7. The method of claim 2, wherein the mutant proto-oncogene is a mutant p53.

8. The method of claim 7, wherein the p53 mutation is a homozygous exon 7 mutation.

9. The method of claim 1, wherein the melanocyte-specific promoter is a nacre-promoter.

10. The method of claim 1, wherein the BRAF mutant is human BRAFV599E mutant.

11. A transgenic zebrafish that expresses, in the fish melanocytes, a mutant human BRAF protein.

12. The transgenic zebrafish of claim 11, wherein the human BRAF protein comprises one or more mutations in its kinase domain.

13. The transgenic zebrafish of claim 11, wherein the human BRAF protein is encoded in the zebrafish by a construct comprising a human BRAF protein encoding sequence operably linked to a melanocyte specific promoter.

14. The transgenic zebrafish of claim 13, wherein the melanocyte promoter is nacre.

15. A transgenic zebrafish expressing a combination of a mutant human BRAF protein and a mutant tumor suppressor protein.

16. The transgenic fish of claim 15, wherein the tumor suppressor protein is mutant p53.

17. The transgenic fish of claim 16, wherein the mutant p53 carried an exon 7 mutation.

18. The transgenic zebrafish of claim 11, wherein expression of the expression product(s) is stable and transmitted through the germline.

19. The transgenic fish of claim 11, wherein expression of the expression products is transient.

20. The transgenic fish according to claims 15, wherein the expression of the mutant tumor suppressor gene is stable and the expression of the mutant BRAF is transient.

21. The method of claim 1, wherein mutant BRAF encoding nucleic acid is transiently expressed.

22. The method of claim 1, wherein mutant BRAF encoding nucleic acid is stably expressed.

23. The method of claim 3 wherein the mutant proto-oncogene is a mutant p53.

24. The method of claim 5 wherein the mutant proto-oncogene is a mutant p53.

25. The method of claim 6 wherein the mutant proto-oncogene is a mutant p53.

26. The transgenic fish according to claims 17, wherein the expression of the mutant tumor suppressor gene is stable and the expression of the mutant BRAF is transient.
